(19) United States
(12) Patent Application Publication
(10) Pub. No.: US 2020/0005926 A1
(43) Pub. Date: Jan. 2, 2020

(54) METHOD AND SYSTEM FOR OPTIMIZING SESSION EXERCISE ORDER OF A PERSONALIZED PRESCRIBED EXERCISE THERAPY PLAN FOR MONITORING COMPLIANCE

(71) Applicant: SWORD HEALTH S.A., PORTO (PT)

(72) Inventors: VIRGÍLIO ANTÓNIO FERRO BENTO, PORTO (PT); IVO EMANUEL MARQUES GABRIEL, PORTO (PT); LUÍS UNGARO PINTO COELHO, PORTO (PT)

(21) Appl. No.: 16/050,282

(22) Filed: Jul. 31, 2018

(30) Foreign Application Priority Data
Jun. 29, 2018 (PT) 110813

(51) Int. Cl. G16H 20/30; A61B 5/11; A61B 5/00; G16H 10/60

(52) U.S. Cl. CPC G16H 20/30; G16H 10/60; A61B 5/7475; A61B 5/1114

(57) ABSTRACT

Methods and a system for managing sessions of physical therapy exercises specific to a patient to maximize outcome are disclosed. Information for each patient includes the sessions to be performed as well as the exercises to be performed within each session. Sensors are attached to specific anatomical landmarks on the patient, depending on the limb to be treated (e.g. hip, shoulder, neck, etc.). The methods and system utilize execution by a data processor to order the exercise data records of the therapy plan data record using input from a monitoring system, store the ordered therapy plan data record, monitor each exercise execution of the patient in real-time according to the ordered therapy plan data record. The method, for instance, can optimize a therapy session for each individual.

### CROSS-REFERENCE TO RELATED APPLICATION

Pursuant to 35 U.S.C. § 119, this application claims the benefit of prior Portugal Application No.: 110813, filed Jun. 29, 2018, which is incorporated herein by reference in its entirety.

### TECHNICAL FIELD

The present disclosure relates to a method and system for optimizing session exercise order of a personalized prescribed exercise therapy plan of a patient for monitoring compliance.

### BACKGROUND

The process of selecting and constantly updating the operational parameters for a medical device capable of applying a therapy is instrumental for the patient's recovery.

Medical devices can responsively provide treatment in accordance with the patient's needs. For example, a sensor can obtain sensed data related to the activity in adjacent tissue, and the sensed data can then be processed to extract features. These features are then evaluated in relation to treatment session ordering, and trigger stimulation treatment in response to defined medically relevant events when these are detected. Sensed activity can also be used to automatically adjust the order of a session protocol according to the features of the detected events or the patient's state. Sensed activity may also be used to responsively provide and adjust an exercise session ordering according to features of the sensed signal.

More specifically, incorrect ordering of exercises of due to human error or lack of medical knowledge might compromise the outcome of the applied therapy. Additionally, many of these devices even require a patient evaluation procedure where the most appropriate session order is determined based upon the measurement of the patient's current condition. The whole process is therefore cumbersome and often compromises a widespread acceptance of the device.

The same principle applies when the exercise ordering is not updated throughout the duration of the treatment.

These facts are disclosed in order to illustrate the technical problem addressed by the present disclosure.

### General Description

The disclosure addresses these issues by selecting and updating the device exercise ordering automatically.

The present disclosure relates to a method and system capable of proving physical therapy sessions to a patient. Such system allows the patient to perform one or more physical therapy sessions, each containing one or more physical therapy exercises. Both the sessions and the exercises are specific to each patient (as they result from the clinical prescription on behalf of his therapist) and usually change throughout the therapy in order to maximize its outcome. The clinical prescription information for each patient thus includes the sessions he must perform as well as the exercises he must perform within each session. Although the prescription does not define a specific number of executions of each session, it does specify the number of executions of each exercise within the session.

The mentioned method and system also requires the patient to attach several sensors to specific anatomical landmarks depending on the limb to be treated (e.g. hip, shoulder, neck, etc.). An important characteristic of exercises pertaining to the same session is that they all share the same placement of the sensors, since each session always aims for the treatment of a single limb.

It is disclosed a method for optimizing session exercise order of a personalized prescribed exercise therapy plan data record for monitoring compliance of a patient, using a monitoring system comprising an electronic data processor, wherein the therapy plan data record comprises one or more session data records, each session data record comprising one or more exercise data records, each exercise data record comprising a prescribed activity requiring physical effort from the patient for the patient; the method comprising the execution by the data processor of: ordering, from a monitoring system user input, the exercise data records of the therapy plan data record, in order; storing the ordered therapy plan data record; monitoring each exercise execution of the patient in real-time according to the ordered therapy plan data record.

It is disclosed a method for optimizing session exercise order of a personalized prescribed exercise therapy plan data record, using a monitoring system comprising an electronic data processor and body part sensors for monitoring compliance of a patient, wherein the therapy plan data record comprises one or more session data records, each session data record comprising a predetermined sensor placement configuration on the patient body and one or more exercise data records, each exercise data record comprising a predetermined body position and a prescribed activity requiring physical effort from the patient;
the method comprising the execution, for sessions not yet marked complete, by the data processor, of the following steps until only one session is selected:
   making a selection of the session or sessions having the lowest number of times the respective session has been completed on the current day;
   keeping in said selection the session or sessions having the same sensor placement in the body as the current sensor placement, if there is already a current sensor placement;
   keeping in said selection the session or sessions having exercise or exercises having the same body position as the current body position, if there is already a current body position;
   keeping in said selection the session or sessions having no exercise having a different body position than the current body position, if there is already a current body position;
   keeping in said selection the session or sessions having exercise or exercises having the body position of standing, if there is already a current body position;
   keeping in the selection any one, and only one, of the selected sessions, if there is still more than one session in said selection;
   displaying to the patient the data comprised in the selected session data record and monitoring the execution of the patient in real-time of the prescribed activity for each exercise for the selected session data record.
   i) An embodiment further comprises, as final selection step, the execution by the data processor of:
keeping in said selection the one, and only one, of the selected sessions that was first recorded for the patient, if there is still more than one session in the selection.

An embodiment further comprises the execution, for exercises not yet marked complete within a current session, by the data processor, of the following steps until only one exercise is selected:
making a selection of the exercise or exercises not having been recorded as completed for the current session;
keeping in the selection the exercise or exercises having the same body position as the current body position, if there is already a current body position;
keeping in the selection the exercise or exercises having the body position of standing, if there is already a current body position;
keeping in the selection any one, and only one, of the selected exercises, if there is still more than one exercise in said selection;
displaying to the patient the data comprised in the selected exercise data record and monitoring the execution of the patient in real-time of the prescribed activity for each exercise data record.

An embodiment further comprises, as final selection step, the execution by the data processor of:
keeping in the selection the one, and only one, of the selected exercises that was first prescribed, if in the last step of the selection, there is still more than one exercise in the selection.

An embodiment further comprises running said exercise selection steps just before an exercise is to be started.

An embodiment further comprises the execution by the data processor of:
marking a session data record as completed if all exercises of the session have been marked as completed.

An embodiment further comprises the execution by the data processor of:
marking an exercise data record as completed if the exercise has been finished or skipped by the user.

An embodiment further comprises running said session selection steps just before a session is to be started.

An embodiment further comprises the execution by the data processor of:
receiving an overriding session input by the patient, just before a session is to be started;
displaying to the patient the data comprised in the overriding session data record and monitoring the execution of the patient in real-time of the prescribed activity for each exercise for the overriding session data record.

An embodiment further comprises the execution by the data processor of:
receiving an exercise skipping input by the patient, during an exercise;
marking the exercise as completed.

An embodiment further comprises the execution by the data processor of:
receiving an interruption input by the patient, during an exercise;
interrupting said monitoring until the patient resumes the monitoring;
resuming monitoring after repeating said exercise selection steps.

It is also disclosed a non-transitory storage media including program instructions for implementing a method for optimizing session exercise order of a personalized prescribed exercise therapy plan data record, using a monitoring system, the program instructions including instructions executable to carry out the method of any of the disclosed embodiments.

It is also disclosed a monitoring system for optimizing session exercise order of a personalized prescribed exercise therapy plan data record, said monitoring system comprising an electronic data processor and body part sensors for monitoring compliance of a patient therapy plan data record, further comprising a non-transitory storage media according to the previous embodiment.

### BRIEF DESCRIPTION OF THE DRAWINGS

The following figures provide preferred embodiments for illustrating the description and should not be seen as limiting the scope of invention.

FIGS. 1A and 1B show a schematic illustration of sensor placement in a first configuration (a) and a second configuration (b), respectively.

FIGS. 2A and 2B show a schematic illustration of two sample exercises, a first standing exercise (a) and a second sitting exercise (b), respectively.

FIG. 3 shows a schematic flow chart of an embodiment for the method for session and exercise selection ordering.

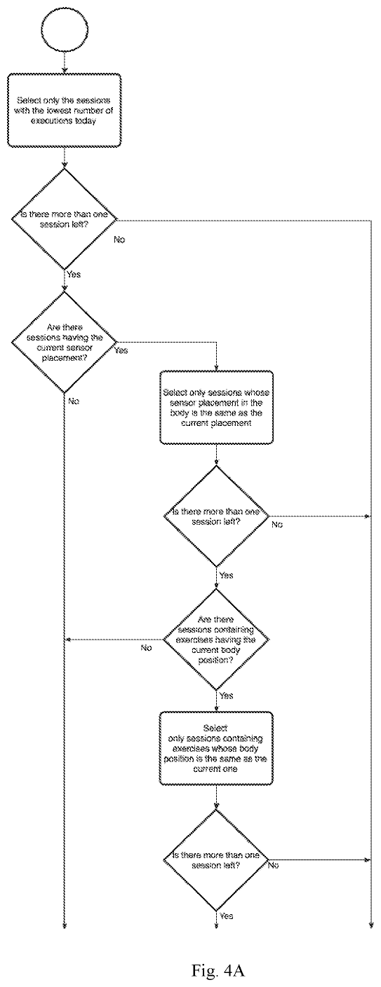

FIGS. 4A and 4B show a schematic flow chart of an embodiment for the method for session selection ordering, wherein the figures are coupled along the connecting match-line arrows.

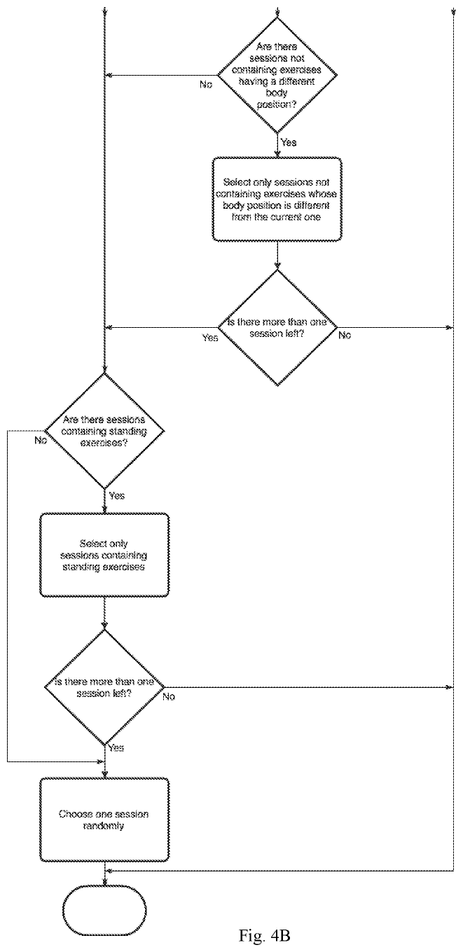

FIG. 5 shows a schematic flow chart of an embodiment for the method for exercise ordering.

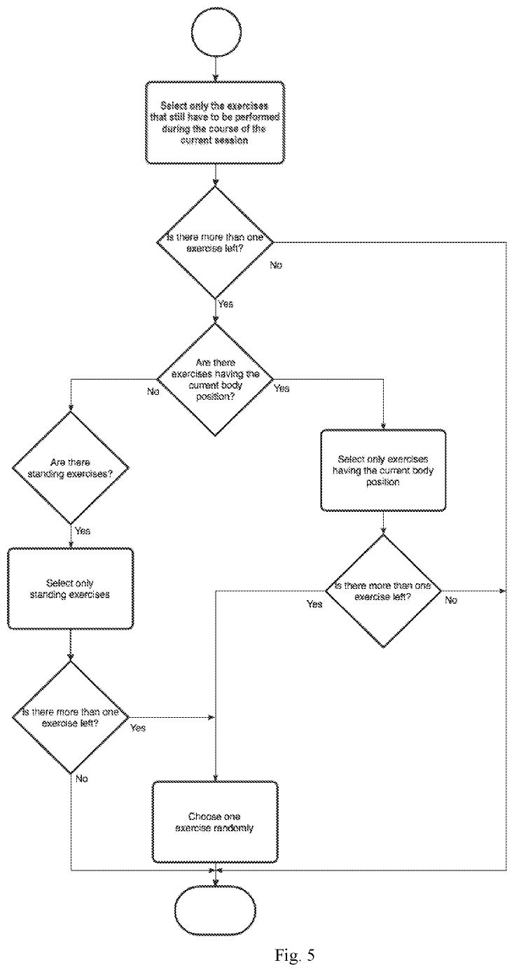

### DETAILED DESCRIPTION

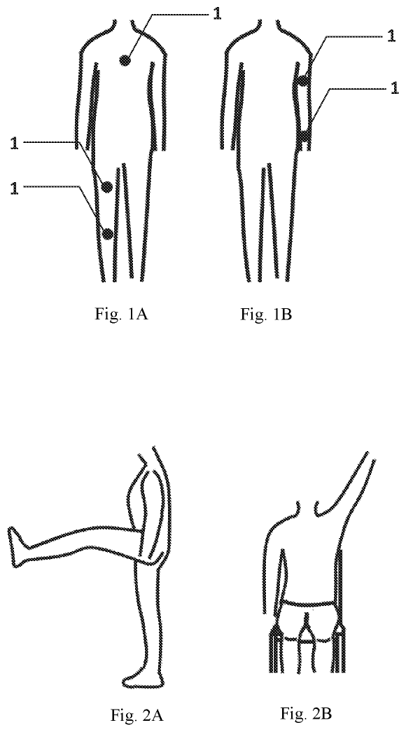

The schematic illustration in FIG. 1 shows how the same session shares the same placement configuration of each sensor (1), since each session aims for the treatment of a single limb or body part (upper leg, upper arm, neck, etc).

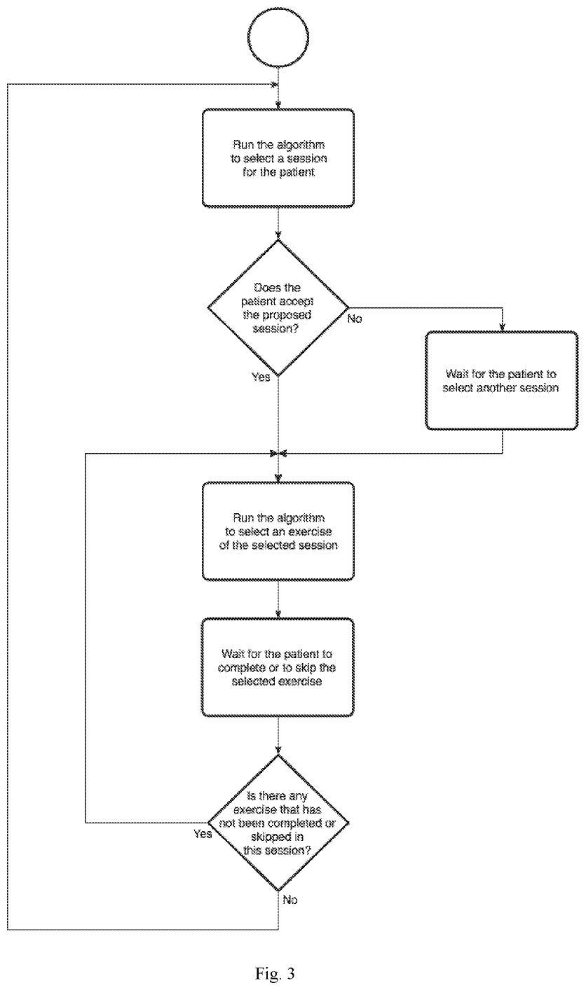

In the present disclosure, exercises in the same session may correspond to different positions of the body (e.g. lying, standing, sitting, etc.). FIG. 2 shows examples of exercises, namely hip flexion (a) and shoulder abduction (b), different positions (standing and seated).

For the purpose of improving the experience of the patient and maximising the therapy's efficiency, it is necessary to perform the sessions and exercises in an appropriate order. In other words, is useful to minimize the number of changes in the placement of the sensors as well as the number of changes in the position of the body while ensuring the therapy is correctly applied.

As such, an algorithm was developed to determine the most appropriate session and exercise ordering.

The obvious solutions, which are those used in these systems are either to have the order input by the therapist while prescribing the sessions or to have standard sets of sessions with a default exercise and session ordering. The drawbacks are the following:
Having only default sets of sessions may be limiting to the effect of the therapy;
Manual ordering on behalf of the therapist will be cumbersome due to the possibly high number of sessions and exercises and will often not be efficient as he will not consider all the required aspects;
Whenever a session is added or an exercise is added to or removed from an existing session the most efficient order may change and the therapist will likely not change the order accordingly;
Having a strict order does not allow the patient to change the order according to his personal convenience (e.g. if the patient does not have time to perform the longest session he may want to perform the shortest one and get back to the largest later).

The developed algorithm does not order the exercises and sessions a priori, instead, it runs each time a new exercise or session is to be started and chooses the most appropriate one according to the current conditions. This is important because it allows the patient to override the ordering in specific situations, while ensuring the best possible ordering after the patient's decision. It also provides the required adaptability to changes in sessions or exercises.

It is important to state that in the main embodiment the patient is allowed to override the automatic session ordering and thus to start, stop and repeat sessions at will but he cannot do the same in regards to the exercises within a session, i.e., the exercise ordering must always be complied with. In other words, the patient is not allowed to select which exercise to perform within a session, it is only allowed to select which session to start. The patient has however, the possibility of skipping any exercise at will, before or during its execution. As such, once a session starts all exercises must be performed according to the default ordering and the session will be marked as done once each of its exercises was either done or skipped.

When selecting the next session to be performed, the algorithm works by ordering the sessions according to a specific set of criteria and selecting the one at the top of the list. In other words, the algorithm starts by ordering all the sessions according to the first criteria, then the algorithm orders the sessions according to the second criteria whilst complying with the first criteria, and so on. An example of such behaviour is the one commonly found in spreadsheet editors or database queries, i.e. ORDER BY column_1, column_2, column_3 etc. where the columns in this case would be the criteria.

As for the actual criteria for ordering the sessions, these are the following:
Number of times performed today in ascending order—sessions undone always have the highest priority as the clinical prescription must be fulfilled
Sessions whose sensor placement in the body is the same as the current placement (if there is one) first—has the purpose of minimising the changes in placement of the sensors
Session containing exercises whose body position is the same as the current one (if there is one) first—has the purpose of minimising the changes in body position
Session not containing exercises whose body position is different from the current one (if there is one) first—the same purpose as the latter
Sessions containing standing exercises first—since in order to setup the system the patient must be in a standing position it always starts by performing exercises in that position

Although the criteria are quite clear, an additional remark must be made regarding the criteria 3 and 4. In fact, the former has the purpose of prioritizing sessions that contain exercises whose position is the same as the current one in order to avoid changes in position. Criterion 4, in turn, has the purpose of selecting, in case there is a tie at criterion 3, sessions that do not contain exercises in other positions because that would minimize the number of changes in setup. The benefits of this criterion become apparent in an example in which the patient is on a lying position and there are two sessions containing lying exercises which have the same performances today and the same setup. If one of these sessions has only lying exercises (session X) and the other has also standing exercises (session Y), if the patient performed session Y first he would have to perform two position changes: the change from lying to standing within session Y and the change from standing to lying position when he finishes session Y and starts session X. If the patient performed session X first, he would only have to perform one change in position: the one within session Y because the transition from session X to session Y would require no changes in position.

The order of the exercises inside a session already in progress, is defined in a similar fashion. The order criteria are as follows:
Exercises that have not been performed yet during the course of the session first—similar to criteria 1 of the session ordering algorithm
Exercises whose body position is the same as the current one (if any) first—same purpose as criteria 3 of the session ordering algorithm
Standing exercises first—same purpose as order criteria 5 of the session ordering algorithm

It can be seen that the above algorithm minimizes the number of changes in the placement of the sensors in the body as well as the number of changes in the position of the body while ensuring the therapy is applied as required by the therapist, thus addressing the presented problem.

As mentioned before, the above algorithm runs every time a new session is to be started, or equivalently, each time the patient starts the system or a session is finished. As such it will continuously react to the last session performed by the patient. The latter also applies to the exercise ordering algorithm, however, as mentioned before, the exercise ordering cannot be overridden.

An example of the algorithm operation could be as follows.

Assume that a patient has the following sessions prescribed and that the following fixed information regarding them applies:

TABLE 1
Sessions prescribed and body positions

| Session ID | Placement of the sensors | Body positions of its exercises |
|---|---|---|
| 1 | Left Upper Leg | Lying, Sitting |
| 2 | Right Upper arm | Lying, Sitting |
| 3 | Right Upper arm | Lying |
| 4 | Left Upper Leg | Sitting |
| 5 | Right Upper arm | Standing, Lying |

Also assume that he starts the system, at a given point in time, with no sensors still placed on his body and that the status of the system is as follows:

TABLE 2
Session and exercise prescribed

| | | Placement | | Session does not |
|---|---|---|---|---|
| | | of the | Session contains | contain exercises |
| | Number of | sensors is the | exercises whose | whose body |
| | Times | same as the | body position | position is |
| Session | Performed | current | is the same as | different from |
| ID | Today | placement | the current one | the current one |
| 1 | 3 | N/A | N/A | N/A |
| 2 | 2 | N/A | N/A | N/A |
| 3 | 2 | N/A | N/A | N/A |
| 4 | 2 | N/A | N/A | N/A |
| 5 | 2 | N/A | N/A | N/A |

According to the algorithm's criteria and the data on the previous tables, the next session to be performed is session 5 because it was the least performed today along with sessions 2, 3 and 4 but in contrast with these last three sessions it has exercises in standing position. It is also important to state the last three columns of the table have N/A values because since the patient has just started the system, he has no sensors on his body (third column) and he has not assumed yet an exercises position (fourth and fifth columns).

Moving forward with our example, let's assume the patient does not reject the suggestion for the next session and performs session 5. When he finishes, he will have the sensors on his right upper arm and will be on the lying position, thus the status of the system will be as follows:

TABLE 3
Session and exercise system status

| | | | Session | Session does |
|---|---|---|---|---|
| | | | contains | not contain |
| | | Placement of | exercises | exercises whose |
| | Number of | the sensors | whose | body position |
| | Times | is the same | body position | is different |
| Session | Performed | as the current | is the same as | from the |
| ID | Today | placement | the current one | current one |
| 1 | 3 | No | Yes | No |
| 2 | 2 | Yes | Yes | No |
| 3 | 2 | Yes | Yes | Yes |
| 4 | 2 | No | No | No |
| 5 | 3 | Yes | Yes | No |

It is clear from the above table that according to the algorithm, the next session to be performed is session 3. However, let's assume the patient overrides the proposed ordering and choses to perform session 1 once more. When he finishes, he will be on a sitting position with the sensors on his left leg. It is clear that the algorithm will suggest that the patient performs session 4 next, thus the algorithm has adapted the output to the patient's decision.

The behavior of the exercise ordering is quite similar, so this example provides also provides insight into how it would work.

1. A method for optimizing session exercise order of a personalized prescribed exercise therapy plan data record, using a monitoring system comprising an electronic data processor and body part sensors for monitoring compliance of a patient, wherein the therapy plan data record comprises one or more session data records, each session data record comprising a predetermined sensor placement configuration on the patient body and one or more exercise data records, each exercise data record comprising a predetermined body position and a prescribed activity requiring physical effort from the patient;
the method comprising the execution, for sessions not yet marked complete, by the data processor, of the following steps until only one session is selected:
making a selection of the session or sessions having the lowest number of times the respective session has been completed on the current day;
keeping in said selection the session or sessions having the same sensor placement in the body as the current sensor placement, if there is already a current sensor placement;
keeping in said selection the session or sessions having exercise or exercises having the same body position as the current body position, if there is already a current body position;
keeping in said selection the session or sessions having no exercise having a different body position than the current body position, if there is already a current body position;
keeping in said selection the session or sessions having exercise or exercises having the body position of standing, if there is already a current body position;
keeping in the selection any one, and only one, of the selected sessions, if there is still more than one session in said selection; and
displaying to the patient the data comprised in the selected session data record and monitoring the execution of the patient in real-time of the prescribed activity for each exercise for the selected session data record.

2. The method according to claim 1, further comprising, as final selection step, the execution by the data processor of:
keeping in said selection the one, and only one, of the selected sessions that was first recorded for the patient, if there is still more than one session in the selection.

3. The method according to claim 1, further comprising the execution, for exercises not yet marked complete within a current session, by the data processor, of the following steps until only one exercise is selected:
making a selection of the exercise or exercises not having been recorded as completed for the current session;
keeping in the selection the exercise or exercises having the same body position as the current body position, if there is already a current body position;
keeping in the selection the exercise or exercises having the body position of standing, if there is already a current body position;
keeping in the selection any one, and only one, of the selected exercises, if there is still more than one exercise in said selection; and
displaying to the patient the data comprised in the selected exercise data record and monitoring the execution of the patient in real-time of the prescribed activity for each exercise data record.

4. The method according to claim 1, further comprising, as final selection step, the execution by the data processor of:
keeping in the selection the one, and only one, of the selected exercises that was first prescribed, if in the last step of the selection, there is still more than one exercise in the selection.

5. The method according to claim 4, further comprising running said exercise selection steps just before an exercise is to be started.

6. The method according to claim 1 further comprising the execution by the data processor of:
marking a session data record as completed if all exercises of the session have been marked as completed.

7. The method according to claim 1, further comprising the execution by the data processor of:
marking an exercise data record as completed if the exercise has been finished or skipped by the user.

8. The method according to claim 7, further comprising running said session selection steps just before a session is to be started.

9. The method according to claim 1, further comprising the execution by the data processor of:
receiving an overriding session input by the patient, just before a session is to be started; and
displaying to the patient the data comprised in the overriding session data record and monitoring the execution of the patient in real-time of the prescribed activity for each exercise for the overriding session data record.

10. The method according to claim 1, further comprising the execution by the data processor of:
receiving an exercise skipping input by the patient, during an exercise;
marking the exercise as completed.

11. The method according to claim 1, further comprising the execution by the data processor of:
receiving an interruption input by the patient, during an exercise; and
interrupting said monitoring until the patient resumes the monitoring;
resuming monitoring after repeating said exercise selection steps.

12. The method according to the claim 1, further comprising the execution, for exercises not yet marked complete within a current session, by the data processor, of the following steps until only one exercise is selected:
making a selection of the exercise or exercises not having been recorded as completed for the current session;
keeping in the selection the exercise or exercises having the same body position as the current body position, if there is already a current body position;
keeping in the selection the exercise or exercises having the body position of standing, if there is already a current body position;
keeping in the selection any one, and only one, of the selected exercises, if there is still more than one exercise in said selection; and
displaying to the patient the data comprised in the selected exercise data record and monitoring the execution of the patient in real-time of the prescribed activity for each exercise data record.

13. The method according to the claim 12, further comprising, as final selection step, the execution by the data processor of:
keeping in the selection the one, and only one, of the selected exercises that was first prescribed, if in the last step of the selection, there is still more than one exercise in the selection.

14. The method according to the claim 13, comprising running said exercise selection steps just before an exercise is to be started.

15. The method according to claim 14, further comprising the execution by the data processor of:
marking a session data record as completed if all exercises of the session have been marked as completed.

16. The method according to the claim 14, further comprising the execution by the data processor of:
marking an exercise data record as completed if the exercise has been finished or skipped by the user.

17. The method according to the claim 1, comprising running said session selection steps just before a session is to be started.

18. The method according to claim 1, further comprising the execution by the data processor of:
receiving an exercise skipping input by the patient, during an exercise; and
marking the exercise as completed.

19. A non-transitory storage media including program instructions for implementing a method for optimizing session exercise order of a personalized prescribed exercise therapy plan data record, using a monitoring system, the program instructions including instructions executable to carry out the method of claim 1.

20. A monitoring system for optimizing session exercise order of a personalized prescribed exercise therapy plan data record, said monitoring system comprising an electronic data processor and body part sensors for monitoring compliance of a patient therapy plan data record, further comprising a non-transitory storage media according to claim 19, wherein said electronic data processor is arranged to carry out the program instructions in said storage media.
